(19) United States
(12) Patent Application Publication
(10) Pub. No.: US 2026/0153802 A1
(43) Pub. Date: Jun. 4, 2026

(54) PHOTOSENSITIVE RESIN COMPOSITION, CURED PRODUCT, AND SEMICONDUCTOR ELEMENT

(71) Applicant: Resonac Corporation, Tokyo (JP)

(72) Inventors: Fumihiko KAWAUCHI, Tokyo (JP); Guillermo FERNANDEZ ZAPICO, Tokyo (JP); Tatsuya MAKINO, Tokyo (JP)

(21) Appl. No.: 18/859,389

(22) PCT Filed: Jan. 18, 2024
(86) PCT No.: PCT/JP2024/001283

§ 371 Date: Oct. 23, 2024

(30) Foreign Application Priority Data
Jan. 19, 2023 (JP) 2023-006398

(51) Int. Cl. G03F 7/028; C07C 211/09; C07C 211/25; H10W 70/695

(52) U.S. Cl. CPC G03F 7/028; C07C 211/09; C07C 211/25; C07C 2601/14; H10W 70/695

(57) ABSTRACT

A photosensitive resin composition according to the present disclosure contains a maleimide compound; a crosslinking agent; and a photopolymerization initiator, in which the maleimide compound is a reaction product of a tetracarboxylic dianhydride (a1), an amine (a2), and maleic anhydride (a3), the amine (a2) includes a dimer diamine and a second amine other than the dimer diamine, and at least one of the tetracarboxylic dianhydride (a1) and the amine (a2) includes a compound having a fluorene skeleton.

### TECHNICAL FIELD

The present disclosure relates to a photosensitive resin composition, a cured product, and a semiconductor element.

### BACKGROUND ART

In accordance with high integration, miniaturization, and micronization of semiconductor elements, insulating films used for surface protective layers, interlayer insulating layers, redistribution layers, and the like of the semiconductor elements are required to have more excellent electrical characteristics, heat resistance, mechanical characteristics, and the like. As a material for forming an insulating film having such characteristics, a photosensitive resin composition containing an alkali-soluble resin has been developed (see, for example, Patent Literatures 1, 2, and 3). These photosensitive resin compositions are applied onto a substrate and dried to form a resin film, and the resin film is exposed and developed to obtain a patterned resin film (a film on which a pattern is formed). Then, a patterned cured film (cured film on which a pattern is formed) can be formed by thermally curing the patterned resin film, and the patterned cured film can be used as an insulating film.

### CITATION LIST

### Patent Literature

Patent Literature 1: JP 2008-309885 A
Patent Literature 2: JP 2007-057595 A
Patent Literature 3: WO 2010/073948 A1

### SUMMARY OF INVENTION

### Technical Problem

A photosensitive resin composition for forming an insulating film of a redistribution layer or the like is required to increase a glass transition temperature (Tg) of the insulating film while sufficiently maintaining fine processability and dielectric characteristics. Therefore, an object of the present disclosure is to provide a photosensitive resin composition capable of forming an insulating film having a high Tg while sufficiently maintaining fine processability and dielectric characteristics.

### Solution to Problem

An aspect of the present disclosure relates to a photosensitive resin composition, a cured product of the photosensitive resin composition, and a semiconductor element described below.

[1] A photosensitive resin composition containing: a maleimide compound; a crosslinking agent; and a photopolymerization initiator, in which the maleimide compound is a reaction product of a tetracarboxylic dianhydride (a1), an amine (a2), and maleic anhydride (a3), the amine (a2) includes a dimer diamine and a second amine other than the dimer diamine, and at least one of the tetracarboxylic dianhydride (a1) and the amine (a2) includes a compound having a fluorene skeleton.

[2] The photosensitive resin composition according to [1], in which the tetracarboxylic dianhydride (a1) includes at least one of 1,3,3a,4,5,9b-hexahydro-5(tetrahydro-2,5-dioxo-3-furanyl)naphtho[1,2-C]furan-1,3-dione, 9,9-bis(3,4-dicarboxyphenyl)fluorene dianhydride, and 9,9-bis[4-(3,4-dicarboxyphenoxy)phenyl]fluorene dianhydride.

[3] The photosensitive resin composition according to [1] or [2], in which the second amine contains at least one of norbornanediamine and 9,9-bis[4-(4-aminophenoxy)phenyl]fluorene.

[4] The photosensitive resin composition according to any one of [1] to [3], in which the dimer diamine contains at least one of a compound represented by the following General Formula (1) and a compound represented by the following General Formula (2):

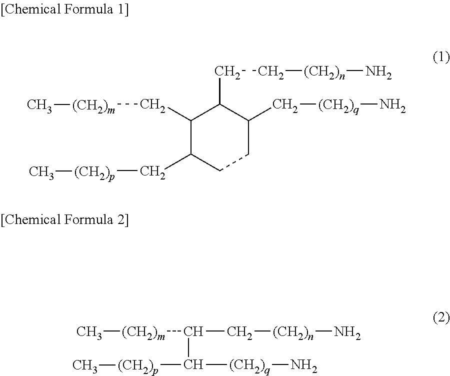

wherein in Formulae (1) and (2), m, n, p, and q each represent an integer of 1 or more selected so that m+n=6 to 17 and p+q=8 to 19, and a bond indicated by a broken line represents a carbon-carbon single bond or a carbon-carbon double bond, and provided that when the bond indicated by the broken line is a carbon-carbon double bond, Formulae (1) and (2) have a structure in which the number of hydrogen atoms bonded to each carbon atom constituting the carbon-carbon double bond is reduced by one from the number indicated in Formulae (1) and (2).

[5] The photosensitive resin composition according to any one of [1] to [4], in which the amine (a2) further includes a triamine.

[6] The photosensitive resin composition according to any one of [1] to [5], in which a weight average molecular weight of the maleimide compound is 3000 to 40000.

[7] The photosensitive resin composition according to any one of [1] to [6], in which the crosslinking agent includes a polymerizable crosslinking agent having a (meth)acryloyl group.

[8] The photosensitive resin composition according to any one of [1] to [7], in which the crosslinking agent includes a polymerizable crosslinking agent having an allyl group or a vinyl group.

[9] The photosensitive resin composition according to any one of [1] to [8], further containing a thermal polymerization initiator.

[10] A cured product of the photosensitive resin composition according to any one of [1] to [9].

[11] A semiconductor element including a redistribution layer containing a cured product of the photosensitive resin composition according to any one of [1] to [9].

### Advantageous Effects of Invention

According to the present disclosure, it is possible to provide a photosensitive resin composition capable of forming an insulating film having a high Tg while sufficiently maintaining fine processability and dielectric characteristics, a cured product having a high Tg while sufficiently maintaining fine processability and dielectric characteristics, and a semiconductor element including a redistribution layer containing the cured product.

### DESCRIPTION OF EMBODIMENTS

Hereinafter, preferred embodiments of the present disclosure will be described in detail. However, the present invention is not limited to the following embodiments, and various modifications can be made within the scope of the gist of the present invention.

In the present specification, a numerical range indicated using “to” indicates a range including numerical values described before and after “to” as a minimum value and a maximum value, respectively. In the numerical range described in stages in the present specification, an upper limit value or a lower limit value of a numerical range of a certain stage can be arbitrarily combined with an upper limit value or a lower limit value of a numerical range of another stage. In the numerical range described in the present specification, the upper limit value or the lower limit value of the numerical range may be replaced with a value shown in Examples. “A or B” may include either A or B, or may include both A and B. The materials exemplified in the present specification can be used alone or in combination of two or more kinds thereof unless otherwise specified. When a plurality of materials corresponding to the respective components are present in the composition, a content of each component in the composition means the total amount of the plurality of materials present in the composition unless otherwise specified.

In the present specification, the “layer” and the “film” include not only a structure having a shape formed on the entire surface but also a structure having a shape formed on a part thereof when observed as a plan view. The term “step” includes not only an independent step but also a step that cannot be clearly distinguished from other steps as long as the intended purpose of the step is achieved.

In the present specification, “(meth)acryloyl” means at least one of “acryloyl” and “methacryloyl” corresponding thereto, and the same applies to other similar expressions such as (meth)acrylic acid and (meth)acrylate. In the present specification, the “solid content” refers to a non-volatile content excluding a volatile substance (water, a solvent, or the like) contained in a photosensitive resin composition, and also includes a component in a liquid, syrupy, or waxy state at room temperature (around 25° C.).

### [Photosensitive Resin Composition]

A photosensitive resin composition according to the present embodiment contains a maleimide compound having a specific structure, a crosslinking agent, and a photopolymerization initiator as essential components. The maleimide compound is a reaction product of a tetracarboxylic dianhydride (a1), an amine (a2), and maleic anhydride (a3), the amine (a2) includes a dimer diamine and a second amine other than the dimer diamine, and at least one of the tetracarboxylic dianhydride (a1) and the amine (a2) includes a compound having a fluorene skeleton.

The photosensitive resin composition according to the present embodiment may further contain a thermal polymerization initiator, a coupling agent, a rust inhibitor, a polymerization inhibitor, and the like, as necessary. The photosensitive resin composition according to the present embodiment is a negative photosensitive resin composition, and a cured product of the photosensitive resin composition can be suitably used as an insulating film for a redistribution layer. Hereinafter, each component used in the photosensitive resin composition of the present embodiment will be described in more detail.

### (Maleimide Compound)

The maleimide compound (hereinafter, also referred to as “component (A)”) according to the present embodiment can be obtained by reacting a tetracarboxylic dianhydride (a1) (hereinafter, also referred to as “component (a1)”), an amine (a2) (hereinafter, also referred to as “component (a2)”), and maleic anhydride (a3) (hereinafter, also referred to as “component (a3)”). That is, the component (A) is a maleimide compound obtained by reacting the component (a1), the component (a2), and the component (a3). Here, the component (a2) includes a dimer diamine and a second amine other than the dimer diamine. In addition, at least one of the component (a1) and the component (a2) includes a compound having a fluorene skeleton. The component (A) may have a plurality of maleimide groups in the molecule. The component (A) may be a bismaleimide compound. The components (A) can be used alone or in combination of two or more kinds thereof.

As the tetracarboxylic dianhydride as the component (a1), those known as a raw material of polyimide can be used. Examples of the component (a1) include pyromellitic anhydride, 4,4′-(hexafluoroisopropylidene)diphthalic anhydride, 1,3,3a,4,5,9b-hexahydro-5(tetrahydro-2,5-dioxo-3-furanyl)naphtho[1,2-C]furan-1,3-dione, 3,3′,4,4′-diphenylsulfonetetracarboxylic dianhydride, 3,3′,4,4′-biphenyltetracarboxylic dianhydride, 3,3′,4,4′-benzophenonetetracarboxylic dianhydride, 4,4′-(4,4′-isopropylidenediphenoxy)diphthalic anhydride, 1,2,3,4-butanetetracarboxylic dianhydride, 1,2,3,4-cyclobutane tetracarboxylic dianhydride, 1,2,3,4-cyclopentane tetracarboxylic dianhydride, 1,2,3,4-tetramethyl-1,2,3,4-cyclobutane tetracarboxylic dianhydride, bicyclo[2.2.2]oct-7-ene-2,3,5,6-tetracarboxylic dianhydride, bis(1,3-dioxo-1,3-dihydroisobenzofuran-5-carboxylic acid) 1,4-phenylene, 9,9-bis(3,4-dicarboxyphenyl)fluorene dianhydride, 4,4′-(ethyne-1,2-diyl)diphthalic anhydride, 5-(2,5-dioxotetrahydrofuryl)-3-methyl-3-cyclohexene-1,2-dicarboxylic anhydride, dicyclohexyl-3,4,3′,4′-tetracarboxylic dianhydride, 3,4′-oxydiphthalic anhydride, 4,4′-oxydiphthalic anhydride, 3,4′-biphthalic anhydride, norbornane-2-spiro-α-cyclopentanone-α′-spiro-2″-norbornane-5,5″,6,6″-tetracarboxylic dianhydride, 5,5′-bis-2-norbornene-5,5′,6,6′-tetracarboxylic acid-5,5′,6,6′-dianhydride, and 9,9-bis[4-(3,4-dicarboxyphenoxy)phenyl]fluorene dianhydride.

From the viewpoint of low dielectric characteristics or a high Tg, the component (a1) preferably contains at least one selected from the group consisting of 1,3,3a,4,5,9b-hexahydro-5(tetrahydro-2,5-dioxo-3-furanyl)naphtho[1,2-C]furan-1,3-dione, 9,9-bis(3,4-dicarboxyphenyl)fluorene dianhydride, 3,3′,4,4′-biphenyltetracarboxylic dianhydride, 4,4′-(4,4′-isopropylidenediphenoxy)diphthalic anhydride, 4,4′-(hexafluoroisopropylidene)diphthalic anhydride, 5-(2,5-dioxotetrahydrofuryl)-3-methyl-3-cyclohexene-1,2-dicarboxylic anhydride, dicyclohexyl-3,4,3′,4′-tetracarboxylic dianhydride, bicyclo[2.2.2]octane-2,3,5,6-tetracarboxylic 2,3:5,6-dianhydride, 5,5′-bis-2-norbornene-5,5′,6,6′-tetracarboxylic acid-5,5′,6,6′-dianhydride, 3,4′-biphthalic anhydride, and 9,9-bis[4-(3,4-dicarboxyphenoxy)phenyl]fluorene dianhydride, more preferably contains at least one selected from the group consisting of 1,3,3a,4,5,9b-hexahydro-5(tetrahydro-2,5-dioxo-3-furanyl)naphtho[1,2-C]furan-1,3-dione, 9,9-bis(3,4-dicarboxyphenyl)fluorene dianhydride, 4,4′-(4,4′-isopropylidenediphenoxy)diphthalic anhydride, 4,4′-(hexafluoroisopropylidene)diphthalic anhydride, and 9,9-bis[4-(3,4-dicarboxyphenoxy)phenyl]fluorene dianhydride, and still more preferably contains at least one of 1,3,3a,4,5,9b-hexahydro-5(tetrahydro-2,5-dioxo-3-furanyl)naphtho[1,2-C]furan-1,3-dione, 9,9-bis(3,4-dicarboxyphenyl)fluorene dianhydride, and 9,9-bis[4-(3,4-dicarboxyphenoxy)phenyl]fluorene dianhydride.

The component (a2) contains a dimer diamine (first amine) and a second amine other than the dimer diamine.

The dimer diamine is, for example, a compound derived from a dimer acid which is a dimer of an unsaturated fatty acid such as oleic acid as described in JP 119-12712 A. By using a dimer diamine as the component (a2), a cured product excellent in dielectric characteristics can be obtained. In the present embodiment, a known dimer diamine can be used without particular limitation. The dimer diamine preferably includes, for example, at least one of a compound represented by the following General Formula (1) and a compound represented by the following General Formula (2).

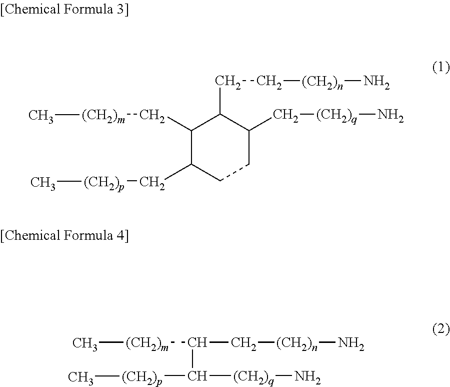

In Formulae (1) and (2), m, n, p, and q each represent an integer of 1 or more selected so that m+n=6 to 17 and p+q=8 to 19, and a bond indicated by a broken line represents a carbon-carbon single bond or a carbon-carbon double bond. Provided that when the bond indicated by the broken line is a carbon-carbon double bond, Formulae (1) and (2) have a structure in which the number of hydrogen atoms bonded to each carbon atom constituting the carbon-carbon double bond is reduced by one from the number indicated in Formulae (1) and (2).

The dimer diamine may be a compound represented by General Formula (2) from the viewpoint of solubility in an organic solvent, heat resistance, heat resistant adhesiveness, low viscosity, and the like, and may be particularly a compound represented by the following Formula (3).

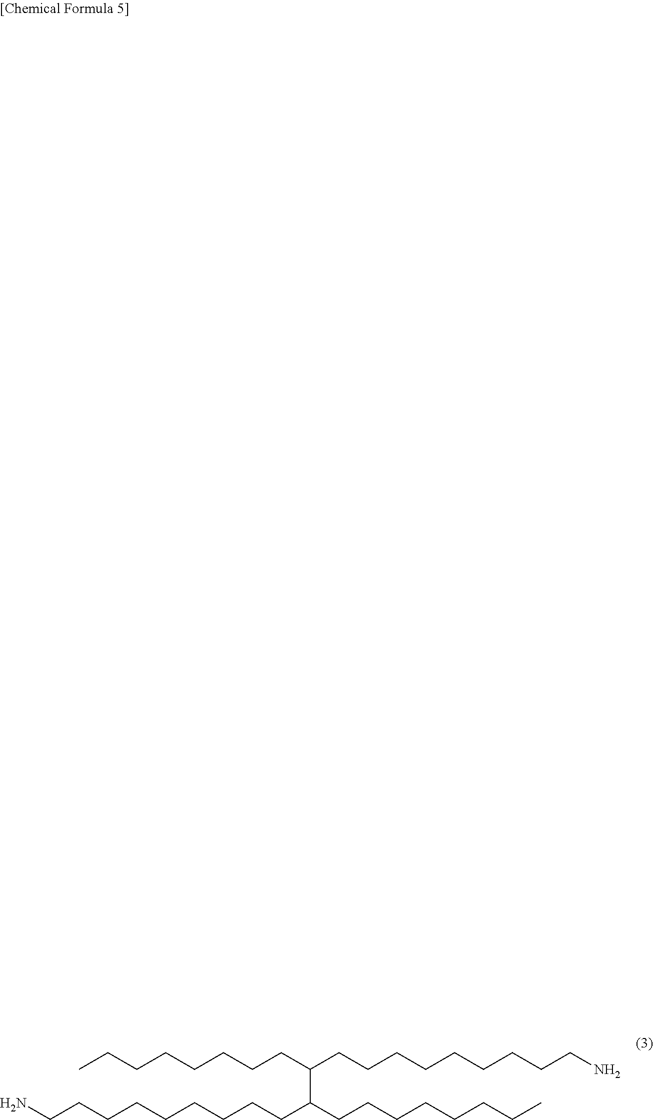

Examples of a commercially available product of the dimer diamine include PRIAMINE 1075 and PRIAMINE 1074 (both manufactured by Croda Japan K.K.).

The second amine is an amine that does not correspond to the dimer diamine described above. The second amine may be a diamine or a triamine, or may be a diamine. By using an alicyclic diamine as the second amine, a dielectric constant can be further reduced. By using an aromatic diamine as the second amine, an elastic modulus and a Tg of the cured product can be improved.

When the second amine is a diamine, examples of the diamine include 1,3-diaminopropane, norbornanediamine, 4,4-methylenedianiline, 1,3-bis[2-(4-aminophenyl)-2-propyl]benzene, 4,4′-diamino-2,2′-bis(trifluoromethyl)biphenyl, 2,2-bis[4-(4-aminophenoxy)phenyl]hexafluoropropane, 9,9-bis(4-aminophenyl)fluorene, 9,9-bis[3-fluoro-4-aminophenyl]fluorene, 9,9-bis[4-(4-aminophenoxy)phenyl]fluorene, 1,3-bis(aminomethyl)cyclohexane, 1,4-bis(aminomethyl)cyclohexane, bis(aminomethyl)norbornane, 4,4′-(hexafluoroisopropylidene)dianiline, 3(4),8(9)-bis(aminomethyl)tricyclo[5.2.1.0^{2,6}]decane, 1,3-cyclohexanediamine, 1,4-cyclohexanediamine, isophoronediamine, 4,4′-methylenebis(cyclohexylamine), 4,4′-methylenebis(2-methylcyclohexylamine), 1,1-bis(4-aminophenyl)cyclohexane, 2,7-diaminofluorene, 4,4′-ethylenedianiline, 4,4′-methylenebis(2,6-diethylaniline), 4,4′-methylenebis(2-ethyl-6-methylaniline), 2,2-bis[4-(4-aminophenoxy)phenyl]propane, bis[4-(4-aminophenoxy)phenyl]methane, 4,4′-bis(4-aminophenoxy)biphenyl, bis[4-(4-aminophenoxy)phenyl]ether, bis[4-(4-aminophenoxy)phenyl]ketone, 1,3-bis(4-aminophenoxy)benzene, 1,4-bis(4-aminophenoxy)benzene, 2,2′-dimethylbiphenyl-4,4′-diamine, (4,4′-diamino)diphenyl ether, (3,3′-diamino)diphenyl ether, paraphenylenediamine, orthophenylenediamine, meta-phenylenediamine, 4,4′-diamino-2,2′-diethylbiphenyl, 4,4′-diamino-3,3′-dimethylbiphenyl, 4,4′-diamino-3,3′-diethylbiphenyl, 4,4′-diamino-3,3′,5,5′-tetramethylbiphenyl, 4,4′-diamino-3,3′,5,5′-tetraethylbiphenyl, 4,4′-diamino-2,2′-dimethoxybiphenyl, 4,4′-diamino-3,3′-dimethoxybiphenyl, meta-xylylenediamine, 4,4′-diaminodiphenylmethane, 4,4′-diaminodiphenyl ether, 3,3′-diaminodiphenyl ether, 1,4′-bis(4-aminophenoxy)benzene, bis[4-(3-aminophenoxy)phenyl]sulfone, and bis[4-(4-aminophenoxy)phenyl]sulfone.

When the second amine is a triamine, examples of the triamine include tris(2-aminomethyl)amine, tris(2-aminoethyl)amine, tris(2-aminopropyl)amine, 2-(aminomethyl)-2-methyl-1,3-propanediamine, a trimer triamine, 3,4,4′-triaminodiphenyl ether, 1,2,4-triaminobenzene, 1,3,5-triaminobenzene, 1,2,3-triaminobenzene, 1,3,5-triazine-2,4,6-triamine, 2,4,6-triaminopyrimidine, 1,3,5-tris(4-aminophenyl)benzene, 1,3,5-tris(4-aminophenoxy)benzene, and tris(4-aminophenyl)methane. Among them, an aliphatic triamine is preferable from the viewpoint of the solubility of the synthesized component (A) in an organic solvent, and tris(2-aminomethyl)amine and tris(2-aminoethyl)amine having a small number of carbon atoms are more preferable from the viewpoint of a high Tg.

The second amine may include one or both of the diamine and the triamine described above. In addition, the second amine may include an amine other than the diamine and the triamine.

From the viewpoint of a high Tg and a high elastic modulus, the second amine preferably contains at least one of norbornanediamine and 9,9-bis[4-(4-aminophenoxy)phenyl]fluorene.

In the component (a2), a molar ratio of the second amine to the total amount of the amines (the number of moles of the second amine/(the number of moles of the dimer diamine+the number of moles of the second amine)) may be 70 mol % or less or 50 mol % or less. When the ratio is 70 mol % or less, a cured product having lower dielectric characteristics can be formed.

When the second amine includes a diamine, in the component (a2), a molar ratio of the diamine in the second amine to the total amount of the diamines (the number of moles of the diamine in the second amine/(the number of moles of the dimer diamine+the number of moles of the diamine in the second amine)) may be 70 mol % or less or 50 mol % or less. When the ratio is 70 mol % or less, a cured product having lower dielectric characteristics can be formed.

By using a dimer diamine as the diamine, a cured product having lower dielectric characteristics can be formed. On the other hand, when only a dimer diamine is used as the diamine, an elastic modulus and a Tg of the cured product decrease. On the other hand, when the second amine is used in combination with a dimer diamine, the elastic modulus and the Tg can be improved while maintaining the dielectric characteristics of the cured product.

At least one of the component (a1) and the component (a2) described above includes a compound having a fluorene skeleton. When at least one of the component (a1) and the component (a2) constituting the maleimide compound includes a compound having a fluorene skeleton, a cured product obtained using the maleimide compound has a high elastic modulus and a high Tg while sufficiently maintaining a low dielectric constant and a low dielectric loss tangent. From the viewpoint of further increasing the elastic modulus and the Tg of the cured product, both the component (a1) and the component (a2) described above may include a compound having a fluorene skeleton.

The component (A) can be prepared by various known methods. For example, first, the component (a1) and the component (a2) are subjected to a polyaddition reaction at a temperature of about 60 to 120° C. and preferably 70 to 90° C., for usually about 0.1 to 2 hours and preferably 0.1 to 1.0 hour. Next, the obtained polyaddition product is further subjected to an imidization reaction, that is, a dehydration ring-closing reaction, at a temperature of about 80 to 250° C. and preferably 100 to 200° C. for about 0.5 to 30 hours and preferably 0.5 to 10 hours. Subsequently, the product obtained by the dehydration ring-closing reaction and the component (a3) are subjected to a maleimidation reaction, that is, a dehydration ring-closing reaction, at a temperature of about 60 to 250° C. and preferably 80 to 200° C. for about 0.5 to 30 hours and preferably 0.5 to 10 hours, thereby obtaining a target component (A).

In the imidization reaction or the maleimidization reaction, various known reaction catalysts, dehydrating agents, and organic solvents can be used.

Examples of the reaction catalyst include aliphatic tertiary amines such as triethylamine, aromatic tertiary amines such as dimethylaniline, heterocyclic tertiary amines such as pyridine, picoline, and isoquinoline, and organic acids such as methanesulfonic acid and p-toluenesulfonic acid monohydrate. Examples of the dehydrating agent include aliphatic acid anhydrides such as acetic anhydride and aromatic acid anhydrides such as benzoic anhydride.

Examples of the organic solvent include aromatic hydrocarbon-based solvents such as benzene, toluene, xylene, mesitylene, and pseudocumene; alcoholic solvents such as methanol, ethanol, isopropyl alcohol, butanol, pentanol, hexanol, propanediol, and phenol; ether-based solvents such as anisole; ketone solvents such as acetone, methyl isobutyl ketone, methyl ethyl ketone, pentanone, hexanone, cyclopentanone, cyclohexanone, isophorone, and acetophenone; cellosolves such as methyl cellosolve and ethyl cellosolve; ester-based solvents such as methyl acetate, ethyl acetate, butyl acetate, methyl propionate, butyl formate, and γ-butyrolactone; glycol ether-based solvents such as ethylene glycol mono-n-butyl ether, ethylene glycol mono-iso-butyl ether, ethylene glycol mono-tert-butyl ether, diethylene glycol mono-n-butyl ether, diethylene glycol mono-iso-butyl ether, triethylene glycol mono-n-butyl ether, and tetraethylene glycol mono-n-butyl ether; and amide-based solvents such as N,N-dimethylformamide, N,N-dimethylacetamide, N-methyl-2-pyrrolidone, N-ethyl-2-pyrrolidone, 3-methoxy-N,N-dimethylpropanamide, and 3-butoxy-N,N-dimethylpropanamide. The organic solvents can be used alone or in combination of two or more kinds thereof.

The component (A) can be purified by various known methods, and the purity can be increased. For example, first, the component (A) dissolved in an organic solvent and pure water are placed in a separatory funnel. Next, the separatory funnel is shaken and allowed to stand. Subsequently, after an aqueous layer and an organic layer are separated, only the organic layer is recovered, such that the component (A) can be purified.

An example of an assumed structure of the component (A) produced by the above method is shown in the following General Formula (4).

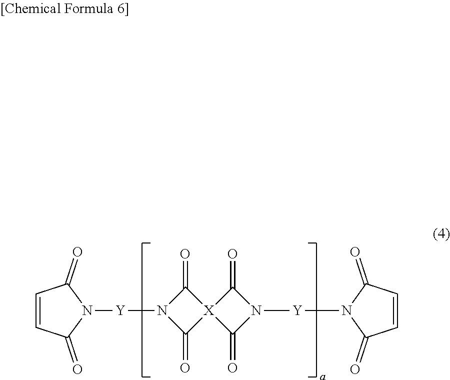

In General Formula (4), X's each independently represent a tetravalent organic group, Y's each independently represent a divalent organic group, and a represents an integer of 1 or more. Provided that at least one of the plurality of Y's represents a divalent organic group derived from the dimer diamine described above, and at least one of the plurality of Y's represents a divalent organic group derived from the second amine (diamine) described above. In addition, X and Y may be an aliphatic group or an organic group having an alicyclic structure or an aromatic ring, which may contain a heteroatom. Provided that at least one of X and Y represents a tetravalent organic group having a fluorene skeleton.

A molecular weight of the component (A) can be controlled by the numbers of moles of the component (a1) and the component (a2), and the molecular weight can be made smaller as the number of moles of the component (a1) is smaller than the number of moles of the component (a2). For the purpose of easily achieving the effect of the present disclosure, the number of moles of the component (a1) with respect to 1 mol of the component (a2), that is, [the number of moles of the component (a1)]/[the number of moles of the component (a2)] is usually in a range of about 0.30 to 1.00, preferably 0.30 to 0.95, more preferably 0.30 to 0.90, and still more preferably 0.50 to 0.80.

The molecular weight of the component (A) may be 3000 or more, 5000 or more, 6000 or more, or 7000 or more, and may be 40000 or less, 38000 or less, 35000 or less, 33000 or less, 30000 or less, 25000 or less, or 20000 or less, in terms of weight average molecular weight (Mw). When the weight average molecular weight is 40000 or less, the solubility in an organic solvent is improved, and when the weight average molecular weight is 3000 or more, an effect of improving heat resistance tends to be sufficiently obtained. From the viewpoint of the solubility in a solvent and heat resistance, the Mw may be 3000 to 40000, and is preferably 3000 to 30000, more preferably 5000 to 25000, still more preferably 6000 to 23000, and particularly preferably 7000 to 20000. The Mw can be measured by gel permeation chromatography (GPC), and can be converted using a calibration curve of standard polystyrene.

### (Crosslinking Agent)

The crosslinking agent (hereinafter, also referred to as “component (B)”) may be a polymerizable crosslinking agent. The polymerizable group may be a photopolymerizable group or a thermopolymerizable group. Examples of the polymerizable group include a (meth)acryloyl group, an allyl group, and a vinyl group. The component (B) may be a polyfunctional compound having two or more polymerizable groups. In addition, the component (B) can crosslink the crosslinking agents and can be crosslinked with the component (A) at the time of exposure of a photosensitive layer, for example. In addition, the component (B) can crosslink the polymerizable crosslinking agents at the time of heating the resin film after pattern formation, for example. The components (B) can be used alone or in combination of two or more kinds thereof.

The resin composition according to the present embodiment may contain a polymerizable crosslinking agent having a (meth)acryloyl group as a crosslinking agent from the viewpoint of dielectric characteristics. The polymerizable crosslinking agent having a (meth)acryloyl group can crosslink the crosslinking agents and can be crosslinked with the component (A) at the time of exposure of the photosensitive layer. The polymerizable crosslinking agent having a (meth)acryloyl group may be an acrylate compound or a methacrylate compound. The component (B) may include a methacrylate compound from the viewpoint of dielectric characteristics.

Examples of the polymerizable crosslinking agent having a (meth)acryloyl group include tricyclodecanedimethanol di(meth)acrylate, tris(2-(meth)acryloyloxyethyl)isocyanurate, dioxane glycol di(meth)acrylate, alkoxylated glycerin tri(meth)acrylate, trimethylolpropane tri(meth)acrylate, alkoxylated trimethylolpropane tri(meth)acrylate, pentaerythritol tri(meth)acrylate, pentaerythritol tetra(meth)acrylate, alkoxylated pentaerythritol tetra(meth)acrylate, 1,6-hexanediol di(meth)acrylate, 1,9-nonanediol di(meth)acrylate, 1,10-decanediol di(meth)acrylate, neopentyl glycol di(meth)acrylate, ethoxylated bisphenol A di(meth)acrylate, propoxylated ethoxylated bisphenol A (meth)acrylate, dipentaerythritol poly(meth)acrylate, alkoxylated dipentaerythritol poly(meth)acrylate, ethoxylated isocyanuric acid tri(meth)acrylate, polyethylene glycol di(meth)acrylate, and polypropylene glycol di(meth)acrylate.

The polymerizable crosslinking agent having a (meth)acryloyl group may include at least one selected from the group consisting of tricyclodecanedimethanol di(meth)acrylate, tris(2-(meth)acryloyloxyethyl)isocyanurate, and dioxane glycol di(meth)acrylate from the viewpoint of heat resistance, dielectric characteristics, and fine processability, and may include tris(2-(meth)acryloyloxyethyl)isocyanurate from the viewpoint of heat resistance and dielectric characteristics.

The resin composition according to the present embodiment may contain, as the crosslinking agent, a polymerizable crosslinking agent having an allyl group or a vinyl group from the viewpoint of dielectric characteristics and heat resistance. The polymerizable crosslinking agent having an allyl group or a vinyl group can crosslink the polymerizable crosslinking agents at the time of heating the resin film after pattern formation.

Examples of the polymerizable crosslinking agent having an allyl group include 1,3,4,6-tetraallyl glycoluril, triallyl isocyanurate, diallyl monoglycidyl isocyanurate, diallyl monomethyl isocyanurate, diallyl isocyanurate, triallyl trimellitate, and triallyl ortho-formate.

Examples of the polymerizable crosslinking agent having a vinyl group include a polyvinyl benzyl compound and a polyvinyl benzyl ether compound.

The polymerizable crosslinking agent having an allyl group or a vinyl group may include at least one selected from the group consisting of 1,3,4,6-tetraallyl glycoluril, triallyl isocyanurate, diallyl isocyanurate, and a polyvinyl benzyl ether compound from the viewpoint of dielectric characteristics and fine processability, and may include 1,3,4,6-tetraallyl glycoluril or triallyl isocyanurate from the viewpoint of dielectric characteristics.

From the viewpoint of further improving a balance between the low dielectric characteristics and the fine processability, a content of the component (B) is preferably less than 50 parts by mass, and may be 1 to 45 parts by mass, 5 to 40 parts by mass, 8 to 30 parts by mass, or 10 to 20 parts by mass, when the total amount of the component (A) and the component (B) is 100 parts by mass.

### (Photopolymerization Initiator)

The photopolymerization initiator (hereinafter, also referred to as “component (C)”) is not particularly limited as long as it is a compound that initiates polymerization by radiation with an active ray (ultraviolet ray or the like), and examples thereof include an alkylphenone-based photopolymerization initiator, an acylphosphine oxide-based photopolymerization initiator, an intramolecular hydrogen abstraction type photopolymerization initiator, and an oxime ester-based photopolymerization initiator.

The alkylphenone-based photopolymerization initiator can be purchased as, for example, Omnirad 651, Omnirad 184, Omnirad 1173, Omnirad 2959, Omnirad 127, Omnirad 907, Omnirad 369, or Omnirad 379EG manufactured by IGM Resins B.V., or the like. The acylphosphine oxide-based photopolymerization initiator can be purchased as, for example, Omnirad 819 or Omnirad TPO H manufactured by IGM Resins B.V., or the like. The intramolecular hydrogen abstraction type photopolymerization initiator can be purchased as, for example, Omnirad MBF or Omnirad 754 manufactured by IGM Resins B.V., or the like. The oxime ester-based photopolymerization initiator can be purchased as, for example, Irgacure OXE01 or Irgacure OXE02 manufactured by BASF Japan Ltd., or the like. In order to promote the photoreaction, a titanocene-based photopolymerization initiator (for example, Irgacure 784, manufactured by BASF Japan Ltd.) may be used in combination.

A content of the component (C) may be 0.1 to 10.0 parts by mass, 0.5 to 8.0 parts by mass, 0.8 to 6.0 parts by mass, or 1.0 to 5.0 parts by mass, with respect to 100 parts by mass of the total amount of the component (A) and the component (B) in terms of easily obtaining excellent fine processability.

### (Thermal Polymerization Initiator)

The photosensitive resin composition according to the present embodiment may further contain a thermal polymerization initiator as the component (D) from the viewpoint of promoting a polymerization reaction of a thermopolymerizable agent. As the component (D), a compound that is decomposed by heating during curing to generate radicals and promotes the polymerization reaction of the component (A) and the component (B) is preferable. Examples of the component (D) include an organic peroxide.

Examples of the organic peroxide include methyl ethyl ketone peroxide, methylcyclohexanone peroxide, methyl acetoacetate peroxide, acetylacetone peroxide, 1,1-bis(t-butylperoxy)-3,3,5-trimethylcyclohexane, 1,1-bis(t-hexylperoxy)cyclohexane, 1,1-bis(t-hexylperoxy)-3,3,5-trimethylcyclohexane, 1,1-bis(t-butylperoxy)cyclohexane, 2,2-bis(4,4-di-t-butylperoxycyclohexyl)propane, 1,1-bis(t-butylperoxy)cyclododecane, n-butyl 4,4-bis(t-butylperoxy)valerate, 2,2-bis(t-butylperoxy)butane, 1,1-bis(t-butylperoxy)-2-methylcyclohexane, t-butyl hydroperoxide, p-menthane hydroperoxide, 1,1,3,3-tetramethylbutyl hydroperoxide, t-hexyl hydroperoxide, dicumyl peroxide, 2,5-dimethyl-2,5-bis(t-butylperoxy)hexane, α,α′-bis(t-butylperoxy)diisopropylbenzene, t-butylcumyl peroxide, di-t-butyl peroxide, 2,5-dimethyl-2,5-bis(t-butylperoxy)hexyne-3, isobutyryl peroxide, 3,5,5-trimethylhexanoyl peroxide, octanoyl peroxide, lauroyl peroxide, cinnamic acid peroxide, m-toluoyl peroxide, benzoyl peroxide, diisopropyl peroxydicarbonate, bis(4-t-butylcyclohexyl) peroxydicarbonate, di-3-methoxybutyl peroxydicarbonate, di-2-ethylhexyl peroxydicarbonate, di-sec-butyl peroxydicarbonate, di(3-methyl-3-methoxybutyl) peroxydicarbonate, di(4-t-butylcyclohexyl)peroxydicarbonate, α,α′-bis(neodecanoylperoxy)diisopropylbenzene, cumylperoxyneodecanoate, 1,1,3,3,-tetramethylbutylperoxyneodecanoate, 1-cyclohexyl-1-methylethyl peroxyneodecanoate, t-hexyl peroxyneodecanoate, t-butyl peroxyneodecanoate, t-hexyl peroxypivalate, t-butyl peroxypivalate, 2,5-dimethyl-2,5-bis(2-ethylhexanoylperoxy)hexane, 1,1,3,3-tetramethylbutylperoxy-2-ethylhexanoate, 1-cyclohexyl-1-methylethylperoxy-2-ethylhexanoate, t-hexylperoxy-2-ethylhexanoate, t-butylperoxy-2-ethylhexanoate, t-butylperoxyisobutyrate, t-butylperoxymaleic acid, t-butylperoxylaurate, t-butylperoxy-3,5,5-trimethylhexanoate, t-butylperoxyisopropyl monocarbonate, t-butylperoxy-2-ethylhexyl monocarbonate, 2,5-dimethyl-2,5-bis(benzoylperoxy)hexane, t-butyl peroxyacetate, t-hexyl peroxybenzoate, t-butyl peroxy-m-toluoyl benzoate, t-butyl peroxybenzoate, bis(t-butylperoxy)isophthalate, t-butylperoxyallyl monocarbonate, and 3,3′,4,4′-tetra(t-butylperoxycarbonyl)benzophenone.

A content of the component (D) is not particularly limited, and may be 0.1 to 10.0 parts by mass, 0.3 to 8.0 parts by mass, 0.5 to 5.0 parts by mass, 0.7 to 3.0 parts by mass, or 0.7 to 2.0 parts by mass, with respect to 100 parts by mass of the total amount of the component (A) and the component (B).

### (Coupling Agent)

The photosensitive resin composition according to the present embodiment may further contain a coupling agent from the viewpoint of improving the adhesion of the cured product of the photosensitive resin composition. The coupling agent may be a silane coupling agent. The silane coupling agent may have, for example, a group such as a vinyl group, an epoxy group, a styryl group, an acryloyl group, a methacryloyl group, an amino group, a ureido group, an isocyanate group, an isocyanurate group, or a mercapto group.

Examples of the silane coupling agent having a vinyl group include KBM-1003 and KBE-1003 (trade name, manufactured by Shin-Etsu Chemical Co., Ltd., the same applies hereinafter). Examples of the silane coupling agent having an epoxy group include KBM-303, 402, and 403, KBE-402 and 403, X-12-981S, and X-12-984S. Examples of the silane coupling agent having a styryl group include KBM-1403. Examples of the silane coupling agent having a methacryloyl group include KBM-502 and 503 and KBE-502 and 503. Examples of the silane coupling agent having an acryloyl group include KBM-5103, X-12-1048, and X-12-1050. Examples of the silane coupling agent having an amino group include KBM-602, 603, 903, 573, and 575, KBE-903 and 9103P, and X-12-972F. Examples of the silane coupling agent having a ureido group include KBE-585. Examples of the silane coupling agent having an isocyanate group include KBE-9007 and X-12-1159L. Examples of the silane coupling agent having an isocyanurate group include KBM-9659. Examples of the silane coupling agent having a mercapto group include KBM-802 and 803, X-12-1154, and X-12-1156. The silane coupling agent may be a silane coupling agent having a methacryloyl group. The silane coupling agents can be used alone or in combination of two or more kinds thereof.

A content of the silane coupling agent may be 0.01 to 10.0 parts by mass, 0.1 to 8.0 parts by mass, 0.3 to 6.0 parts by mass, 0.5 to 5.0 parts by mass, or 1.0 to 3.0 parts by mass, with respect to 100 parts by mass of the total amount of the component (A) and the component (B).

### (Rust Inhibitor)

The photosensitive resin composition according to the present embodiment may further contain a rust inhibitor from the viewpoint of suppressing corrosion of the copper wiring or preventing discoloration. Examples of the rust inhibitor include a triazole derivative such as benzotriazole and a tetrazole derivative. The rust inhibitors may be used alone or in combination of two or more kinds thereof.

A content of the rust inhibitor may be 0.01 to 10.0 parts by mass, 0.1 to 5.0 parts by mass, 0.3 to 4.0 parts by mass, 0.5 to 3.0 parts by mass, or 1.0 to 3.0 parts by mass, with respect to 100 parts by mass of the total amount of the component (A) and the component (B).

### (Polymerization Inhibitor)

The photosensitive resin composition according to the present embodiment may further contain a polymerization inhibitor from the viewpoint of storage stability.

Examples of the polymerization inhibitor include 4-tert-butylcatechol, 4-hydroxy-2,2,6,6-tetramethylpiperidine-1-oxy radical, p-methoxyphenol, diphenyl-p-benzoquinone, benzoquinone, hydroquinone, pyrogallol, phenothiazine, resorcinol, ortho-dinitrobenzene, para-dinitrobenzene, meta-dinitrobenzene, phenanthraquinone, N-phenyl-2-naphthylamine, cupferron, 2,5-toluquinone, tannic acid, para-benzylaminophenol, tris(4-tert-butyl-3-hydroxy-2,6-dimethylbenzyl)isocyanuric acid, and nitrosamines. The polymerization inhibitors may be used alone or in combination of two or more kinds thereof.

A content of the polymerization inhibitor may be 0.01 to 10.0 parts by mass, 0.05 to 5.0 parts by mass, 0.10 to 2.0 parts by mass, or 0.10 to 1.0 part by mass, with respect to 100 parts by mass of the total amount of the component (A) and the component (B).

### (Sensitizer)

The photosensitive resin composition may further contain a sensitizer from the viewpoint of achieving both maintenance of a residual film ratio in a wide range of exposure amount and excellent resolution.

Examples of the sensitizer include Michler's ketone, benzoin, 2-methylbenzoin, benzoin methyl ether, benzoin ethyl ether, benzoin isopropyl ether, benzoin butyl ether, 2-t-butylanthraquinone, 1,2-benzo-9,10-anthraquinone, anthraquinone, methylanthraquinone, 4,4′-bis(diethylamino)benzophenone, acetophenone, benzophenone, thioxanthone, 1,5-acenaphthene, 2,2-dimethoxy-2-phenylacetophenone, 1-hydroxycyclohexyl phenyl ketone, 2-methyl-[4-(methylthio)phenyl]-2-morpholino-1-propanone, diacetyl benzyl, benzyldimethyl ketal, benzyldiethyl ketal, diphenyl disulfide, anthracene, phenanthrene quinone, riboflavin tetrabutyrate, acridine orange, erythrosine, phenanthrenequinone, 2-isopropylthioxanthone, 2,6-bis(p-diethylaminobenzylidene)-4-methyl-4-azacyclohexanone, 6-bis(p-dimethylaminobenzylidene)-cyclopentanone, 2,6-bis(p-diethylaminobenzylidene)-4-phenylcyclohexanone, aminostyryl ketone, a 3-ketocoumarin compound, a biscoumarin compound, N-phenylglycine, N-phenyldiethanolamine, and 3,3′,4,4′-tetra(t-butylperoxycarbonyl)benzophenone. The sensitizers may be used alone or in combination of two or more kinds thereof.

When the photosensitive resin composition contains a sensitizer, a content thereof is preferably 0.1 to 2.0 parts by mass, and more preferably 0.2 to 1.5 parts by mass, with respect to 100 parts by mass of the total mass of the component (A) and the component (B).

### (Solvent)

When the photosensitive resin composition according to the present embodiment contains a solvent for dissolving and dispersing each component, the photosensitive resin composition can be easily applied onto a substrate to form a coating film having a uniform thickness. The solvents may be used alone or in combination of two or more kinds thereof.

Examples of the solvent include ketone-based solvents such as methyl ethyl ketone, cyclohexanone, and cyclopentanone; aromatic hydrocarbon-based solvents such as toluene, xylene, tetramethylbenzene, mesitylene, and pseudocumene; glycol ether-based solvents such as methyl cellosolve, butyl cellosolve, methyl carbitol, butyl carbitol, propylene glycol monomethyl ether, dipropylene glycol monoethyl ether, dipropylene glycol dimethyl ether, dipropylene glycol diethyl ether, and triethylene glycol monoethyl ether; ester-based solvents such as ethyl acetate, butyl acetate, butyl cellosolve acetate, carbitol acetate, and γ-butyrolactone; and amide-based solvents such as N,N-dimethylformamide, N,N-dimethylacetamide, N-methyl-2-pyrrolidone, N-ethyl-2-pyrrolidone, 3-methoxy-N,N-dimethylpropanamide, and 3-butoxy-N,N-dimethylpropanamide.

A blending amount of the solvent is not particularly limited, and may be an amount in which a solid content in the photosensitive resin composition is 5 to 60 mass %, 10 to 50 mass %, or 15 to 40 mass %.

The preparation means, conditions, and the like of the photosensitive resin composition are not particularly limited. Examples thereof include a method in which the respective main components are sufficiently uniformly stirred and mixed in predetermined blending amounts by a mixer or the like, and then kneaded by using a mixing roll, an extruder, a kneader, a roll, an extruder, or the like. The kneading method is not particularly limited.

A relative dielectric constant of the cured product of the photosensitive resin composition according to the present embodiment at 10 GHz may be 2.80 or less, 2.75 or less, or 2.70 or less. A dielectric loss tangent of the cured product of the photosensitive resin composition at 10 GHz may be 0.0060 or less, 0.0050 or less, 0.0045 or less, or 0.0040 or less. The relative dielectric constant and the dielectric loss tangent can be measured by the method described in Examples using a cured film of the photosensitive resin composition.

The photosensitive resin composition according to the present embodiment can form a fine pattern. The photosensitive resin composition according to the present embodiment can form an insulating film exhibiting low dielectric characteristics and excellent insulation reliability. A semiconductor element including an interlayer insulating layer formed using a cured product of the photosensitive resin composition described above, and an electronic device including the semiconductor element can be produced. The semiconductor element includes a redistribution layer containing the cured product of the photosensitive resin composition according to the present embodiment, such that high frequency characteristics can be improved. The semiconductor element may be, for example, a memory, a package, or the like having a multilayer wiring structure, a redistribution structure, or the like. Examples of the electronic device include a mobile phone, a smartphone, a tablet terminal, a personal computer, and a hard disk suspension. By including a patterned cured film formed of the photosensitive resin composition of the present embodiment, it is possible to provide a semiconductor element and an electronic device having excellent reliability.

### EXAMPLES

Hereinafter, the present disclosure will be described in more detail with reference to Examples, but the present invention is not limited to these Examples.

### Synthesis Example 1

Into a 0.3 L flask vessel equipped with a condenser, a nitrogen introducing tube, a thermocouple, and a stirrer, 26.27 parts by mass of 9,9-bis(3,4-dicarboxyphenyl)fluorene dianhydride (trade name “BPAF”, manufactured by JFE Chemical Corporation), 120.56 parts by mass of T-SOL 100 (trade name, manufactured by ENEOS Corporation, aromatic high-boiling solvent), and 32.15 parts by mass of SOLMIX A-11 (trade name, manufactured by Japan Alcohol Trading Co., Ltd., alcoholic solvent) were introduced. After the introduction, the temperature was raised to 80° C., the mixture was kept warm for 0.5 hours, and 28.20 parts by mass of a dimer diamine (trade name: “PRIAMINE 1075”, manufactured by Croda Japan K.K.) was added dropwise. After the dropwise addition, 1.40 parts by mass of tris(2-aminoethyl)amine (manufactured by Tokyo Chemical Industry Co., Ltd.) and 4.42 parts by mass of norbornanediamine (manufactured by Mitsui Fine Chemicals, Inc.) were added dropwise in this order, and after the dropwise addition, the mixture was kept warm at 80° C. for 0.5 hours. After keeping the mixture warm, 3.41 parts by mass of a methanesulfonic acid aqueous solution (trade name “Lutropur MSA”, manufactured by BASF Japan Ltd.) was added. Thereafter, the temperature was raised to 160° C. while removing the alcohol in the reaction solution. After raising the temperature, 40.00 parts by mass of toluene (manufactured by Yamaichi Chemical Industries Co., Ltd.) was added, and a dehydration ring-closing reaction was performed at 160° C. for 2 hours to remove water and alcohol in the reaction solution, thereby obtaining an intermediate polyimide resin. Subsequently, the obtained polyimide resin was cooled to 130° C., 11.24 parts by mass of maleic anhydride (manufactured by FUSO CHEMICAL CO., LTD.) was added, the temperature was raised to 160° C., a dehydration ring-closing reaction was performed at 160° C. for 4 hours, and water in the reaction solution was removed, thereby obtaining a maleimide compound.

The obtained maleimide compound was placed in a separatory funnel, 500 parts by mass of pure water was added thereto, and the separatory funnel was shaken and allowed to stand. After the standing, an aqueous layer and an organic layer were separated, and then only the organic layer was recovered. The recovered organic layer was introduced into a 0.3 L glass vessel equipped with a condenser, a nitrogen introducing tube, a thermocouple, a stirrer, and a vacuum pump, the temperature was raised to 88 to 93° C., and then, water was removed. Thereafter, the temperature was raised to 100° C., and the solvent was partially removed for 0.5 hours in a state where the pressure was reduced from the atmospheric pressure by 0.1 MPa, thereby obtaining a solution of a maleimide compound (A-1) as a component (A).

### Synthesis Example 2

A solution of a maleimide compound (A-2) was obtained in the same manner as that of Synthesis Example 1, except that tris(2-aminoethyl)amine was not used and the blending amounts of the respective components were changed as shown in Table 1.

### Synthesis Example 3

A solution of a maleimide compound (A-3) was obtained in the same manner as that of Synthesis Example 1, except that tris(2-aminoethyl)amine was not used and the blending amounts of the respective components were changed as shown in Table 1.

### Synthesis Example 4

A solution of a maleimide compound (A-4) was obtained in the same manner as that of Synthesis Example 1, except that BPAF was changed to 1,3,3a,4,5,9b-hexahydro-5(tetrahydro-2,5-dioxo-3-furanyl)naphtho[1,2-C]furan-1,3-dione (trade name “TDA-100”, manufactured by New Japan Chemical Co., Ltd.), the norbornanediamine was changed to 9,9-bis[4-(4-aminophenoxy)phenyl]fluorene (trade name “BPF-AN”, manufactured by JFE Chemical Corporation), tris(2-aminoethyl)amine was not used, and the blending amounts of the respective components were changed as shown in Table 1.

### Synthesis Example 5

A solution of a maleimide compound (A-5) was obtained in the same manner as that of Synthesis Example 1, except that tris(2-aminoethyl)amine was not used, T-SOL 100 was changed to pseudocumene (manufactured by Toyo Gosei Co., Ltd., aromatic high-boiling solvent) and N-methyl-2-pyrrolidone (NMP), and the blending amounts of the respective components were changed as shown in Table 1.

### (Non-Volatile Content)

0.75 g±0.25 g of the solution of the maleimide compound was weighed on a metal petri dish with a precision balance, and then dried at 150° C. for 0.5 hours with a hot air dryer, and the non-volatile content (NV) was calculated using the following equation.

$$NV ( mass \% ) = \{ ( W 3 - W 1 ) / W 2 \} \times 100$$

W1: mass (g) of empty metal petri dish
W2: mass (g) of solution of maleimide compound before drying
W3: mass (g) of metal petri dish+maleimide compound after drying

### (Weight Average Molecular Weight)

A weight average molecular weight (Mw) of the maleimide compound was measured by gel permeation chromatography (GPC). 50 μL of a sample obtained by dissolving the maleimide compound in tetrahydrofuran (THF) so as to have a concentration of 3 mass % was injected into columns (GL-R420×1, GL-R430×1, and GL-R440×1 (all columns are manufactured by Hitachi High-Tech Fielding Corporation)) heated to 30° C., and the measurement was performed under the condition of a flow rate of 1.6 mL/min using THF as a developing solvent. Note that, as a detector, L-3350 RI detector (manufactured by Hitachi, Ltd.) was used, and the Mw was converted from the elution time by a molecular weight/elution time curve created using standard polystyrene (manufactured by Tosoh Corporation).

TABLE 1

| | | A-1 | A-2 | A-3 | A-4 | A-5 |
|---|---|---|---|---|---|---|
| Component | BPAF | 26.27 | 39.39 | 39.39 | — | 111.79 |
| (a1) | TDA-100 | — | — | — | 25.78 | — |
| Component | Dimer diamine | 28.20 | 43.07 | 30.76 | 30.75 | 104.70 |
| (a2) | Norbornanediamine | 4.42 | 5.30 | 8.83 | — | 20.02 |
| | Tris(2-aminoethyl)amine | 1.40 | — | — | — | — |
| | BPF-AN | — | — | — | 30.51 | — |
| Component | Maleic anhydride | 11.24 | 8.42 | 8.42 | 8.42 | 23.90 |
| (a3) | | | | | | |
| Acid | Methanesulfonic acid | 3.41 | 2.20 | 2.20 | 2.20 | 4.40 |
| catalyst | aqueous solution | | | | | |
| Solvent | T-SOL | 120.56 | 165.77 | 152.06 | 164.87 | — |
| | Pseudocumene | — | — | — | — | 250.10 |
| | SOLMIX A-11 | 32.15 | 35.04 | 32.69 | 35.69 | 107.40 |
| | NMP | — | — | — | — | 50.00 |
| | Toluene | 40.00 | 50.00 | 50.00 | 90.00 | 60.00 |

| | | | | | |
|---|---|---|---|---|---|
| NV (mass %) | 47.6 | 56.0 | 47.2 | 46.0 | 57.0 |
| Mw | 13600 | 12300 | 11200 | 8800 | 9700 |

The following compounds were prepared as components (B).
A-9300: tris(2-acryloyloxyethyl)isocyanurate (trade name, manufactured by SHIN-NAKAMIURA CHEMICAL CO., LTD.)
TA-G: 1,3,4,6-tetraallyl glycoluril (trade name, manufactured by SHIKOKU CHEMICALS CORPORATION)
TAIC: triallyl isocyanurate (trade name, manufactured by Shinryo Corporation)

The following compounds were prepared as the component (C), the component (D), the coupling agent, the polymerization inhibitor, the rust inhibitor, and the solvent.
Component (C): oxime ester-based photopolymerization initiator (trade name: “Irgacure OXE01” and “Irgacure OXE02”, manufactured by BASF Japan Ltd.)
Component (D): α,α′-bis(t-butylperoxy)diisopropylbenzene (trade name: “PERBUTYL P”, manufactured by NOF CORPORATION)
Coupling agent: 3-methacryloxypropyltrimethoxysilane (trade name “KBM-503”, manufactured by Shin-Etsu Chemical Co., Ltd.)
Polymerization inhibitor: 4-hydroxy-2,2,6,6-tetramethylpiperidine-1-oxy radical (TEMPOL) (manufactured by Tokyo Chemical Industry Co., Ltd.)
Rust inhibitor: 1,2,3-benzotriazole (trade name “BT-120”, manufactured by Johoku Chemical CO., LTD.)
Solvent: mesitylene (manufactured by Toyo Gosei Co., Ltd.)

### [Photosensitive Resin Composition]

The respective components in the blending amounts (parts by mass, solid content) shown in Table 2 and 210 parts by mass of a solvent were mixed, and the mixture was stirred at 25° C. for 30 minutes or longer and then filtered through a filter with a mesh size of 0.5 μm, thereby preparing photosensitive resin compositions of Examples 1 to 8.

### (Dielectric Characteristics)

The photosensitive resin composition was applied onto a copper foil using a knife coater, air-dried for 15 minutes, and dried in a dryer at 90° C. for 15 minutes to form a coating film. The coating film was exposed to light from a high-pressure mercury lamp (exposure amount: 1000 mJ/cm²) and then baked (100° C., 1 minute) on a hot plate after exposure to light to form a resin film having a thickness of 100 μm. Thereafter, the resin film was cured at 200° C. for 2 hours using a clean oven in a nitrogen atmosphere. Subsequently, the copper foil was dissolved and removed with ammonium persulfate to obtain a cured film.

The cured film was cut into a length of 80 mm and a width of 80 mm to prepare an evaluation sample. A relative dielectric constant (Dk) and a dielectric loss tangent (Df) of the evaluation sample at 10 GHz were measured at room temperature using an SPDR dielectric resonator (manufactured by QWED Company) and an analyzer (trade name “PNA Network Analyzer N5227A”, manufactured by Agilent Technologies, Inc.).

### (Mechanical Characteristics)

The cured film was cut into a length of 40 mm and a width of 2 mm to prepare an evaluation sample. The evaluation sample was measured using a thermomechanical analyzer (trade name “Q-400”, manufactured by TA Instruments Japan Inc.) in a nitrogen atmosphere under the conditions of a tensile mode, a load of 5 mN, a measurement temperature range of −50 to 220° C., a heating rate of 10° C./min, and a distance between chucks of 10 mm, and a coefficient of thermal expansion (CTE) was calculated from a displacement at 0 to 40° C.

### (5% Weight Loss Temperature)

6.0 to 10.0 mg of the cured film was weighed in an open type sample container (trade name “GCA-0055”, manufactured by Hitachi High-Tech Science Corporation), and a 5% weight loss temperature (Tas) was measured under the conditions of a nitrogen flow rate of 300 mL/min, a starting temperature of 40° C., and a heating rate of 10° C./min. As a measuring apparatus, NEXTA STA200RV (manufactured by Hitachi High-Tech Science Corporation) was used.

### (Tg)

The photosensitive resin composition was spin-coated onto a silicon wafer with a Cu sputtered film, and dried by heating at 90° C. for 5 minutes to form a resin film having a thickness of 14 μm. Next, the resin film was subjected to pattern exposure under the condition of an exposure amount of 1000 mJ/cm² using a mask aligner-exposure machine (MA-20, manufactured by MIKASA CO., LTD.) so as to have a width of 10 mm, and then the resin film was heated at 100° C. for 1 minute. The resin film after exposure was developed using a developer (a mixed solution of cyclopentanone and propylene glycol monomethyl ether acetate) at 25° C. for 30 seconds, and then washed with propylene glycol monomethyl ether acetate, and the resin film was cured at 200° C. for 2 hours in a nitrogen atmosphere. The silicon wafer on which the patterned cured film was formed was immersed in an ammonium persulfate aqueous solution, and the cured film was peeled off from the silicon wafer. The cured film was washed with pure water to obtain a strip-shaped resin film having a width of 10 mm. The resin film was cut into a length of 50 mm, and a Tg was measured using a dynamic viscoelasticity measuring device (trade name “RSA G2”, manufactured by TA Instruments Japan Inc.) under the conditions of a distance between chucks of 20 mm and a measurement temperature range of −50 to 350° C.

### (Fine Processability)

The photosensitive resin composition was spin-coated onto a silicon wafer, and dried by heating at 90° C. for 5 minutes using a hot plate to form a resin film having a thickness of 7 μm. Next, the resin film was subjected to pattern exposure under the condition of an exposure amount of 300 mJ/cm² using an i-line stepper exposure machine (trade name “Sc6k”, manufactured by CERMA PRECISION, INC.), and then heated at 100° C. for 1 minute using a hot plate. Thereafter, the film was developed using a developer (a mixed solution of cyclopentanone and propylene glycol monomethyl ether acetate) at 25° C. for 30 seconds (2 times for 15 seconds), and washed with propylene glycol monomethyl ether acetate. The presence or absence of peeling of the resin film after development from the silicon wafer, cracking of the resin film, or roughness of the patterned end portion, and the presence or absence of a residue of the patterned bottom portion obtained by developing the resin film were confirmed with a metal microscope. A case where the minimum via diameter in which these defects were not confirmed was 20 μm or less was evaluated as “A”, and a case where the minimum via diameter was more than 20 μm was evaluated as “B”.

TABLE 2

| Example | | 1 | 2 | 3 | 4 | 5 | 6 | 7 | 8 | 9 |
|---|---|---|---|---|---|---|---|---|---|---|
| Component | A-1 | 85.0 | 85.0 | 85.0 | 85.0 | 85.0 | — | — | — | — |
| (A) | A-2 | — | — | — | — | — | 85.0 | — | — | — |
| | A-3 | — | — | — | — | — | — | 85.0 | — | — |
| | A-4 | — | — | — | — | — | — | — | 85.0 | — |
| | A-5 | — | — | — | — | — | — | — | — | 85.0 |
| Component | A-9300 | 15.0 | 10.0 | 7.5 | 5.0 | — | 15.0 | 15.0 | 15.0 | 5.0 |
| (B) | TA-G | — | 5.0 | 7.5 | 10.0 | 15.0 | — | — | — | — |
| | TAIC | — | — | — | — | — | — | — | — | 10.0 |
| Component | OXE01 | 3.0 | 3.0 | 3.0 | 3.0 | 3.0 | 3.0 | 3.0 | 3.0 | 3.0 |
| (C) | OXE02 | — | — | — | — | — | — | — | — | 2.0 |

| | | | | | | | | | |
|---|---|---|---|---|---|---|---|---|---|
| Component (D) | 1.0 | 1.0 | 1.0 | 1.0 | 1.0 | 1.0 | 1.0 | 1.0 | — |
| Coupling agent | 1.7 | 1.7 | 1.7 | 1.7 | 1.7 | 1.7 | 1.7 | 1.7 | 1.7 |
| Polymerization | 0.25 | 0.25 | 0.25 | 0.25 | 0.25 | 0.25 | 0.25 | 0.25 | 0.25 |
| inhibitor | | | | | | | | | |
| Rust inhibitor | 1.7 | 1.7 | 1.7 | 1.7 | 1.7 | 1.7 | 1.7 | 1.7 | 1.7 |
| Dk | 2.67 | 2.70 | 2.69 | 2.71 | 2.69 | 2.66 | 2.72 | 2.75 | 2.50 |
| Df | 0.0041 | 0.0038 | 0.0037 | 0.0034 | 0.0031 | 0.0038 | 0.0043 | 0.0047 | 0.0018 |
| Td5 (° C.) | 412 | 412 | 415 | 415 | 421 | 410 | 410 | 410 | 423 |
| CTE (ppm/° C.) | 97 | 99 | 101 | 99 | 99 | 111 | 97 | 95 | 81 |
| Tg (° C.) | 140 | 140 | 138 | 135 | 133 | 110 | 157 | 164 | 142 |
| Via diameter | A | A | A | A | A | A | A | A | A |
| evaluation | | | | | | | | | |

1. A photosensitive resin composition comprising: a maleimide compound; a crosslinking agent; and a photopolymerization initiator,
wherein the maleimide compound is a reaction product of a tetracarboxylic dianhydride (a1), an amine (a2), and maleic anhydride (a3),
the amine (a2) includes a dimer diamine and a second amine other than the dimer diamine, and
at least one of the tetracarboxylic dianhydride (a1) and the amine (a2) includes a compound having a fluorene skeleton.

2. The photosensitive resin composition according to claim 1, wherein the tetracarboxylic dianhydride (a1) includes at least one of 1,3,3a,4,5,9b-hexahydro-5(tetrahydro-2,5-dioxo-3-furanyl)naphtho[1,2-C]furan-1,3-dione, 9,9-bis(3,4-dicarboxyphenyl)fluorene dianhydride, and 9,9-bis[4-(3,4-dicarboxyphenoxy)phenyl]fluorene dianhydride.

3. The photosensitive resin composition according to claim 1, wherein the second amine contains at least one of norbornanediamine and 9,9-bis[4-(4-aminophenoxy)phenyl]fluorene.

4. The photosensitive resin composition according to claim 1, wherein the dimer diamine contains at least one of a compound represented by the following General Formula (1) and a compound represented by the following General Formula (2): 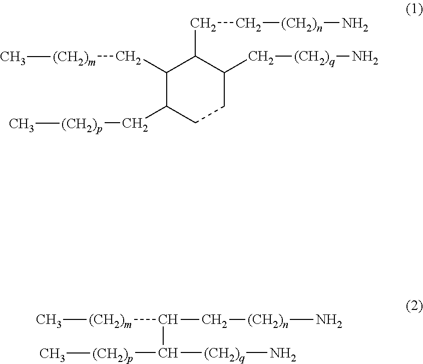
wherein in Formulae (1) and (2), m, n, p, and q each represent an integer of 1 or more selected so that m+n=6 to 17 and p+q=8 to 19, and a bond indicated by a broken line represents a carbon-carbon single bond or a carbon-carbon double bond, and provided that when the bond indicated by the broken line is a carbon-carbon double bond, Formulae (1) and (2) have a structure in which the number of hydrogen atoms bonded to each carbon atom constituting the carbon-carbon double bond is reduced by one from the number indicated in Formulae (1) and (2).

5. The photosensitive resin composition according to claim 1, wherein the amine (a2) further includes a triamine.

6. The photosensitive resin composition according to claim 1, wherein a weight average molecular weight of the maleimide compound is 3000 to 40000.

7. The photosensitive resin composition according to claim 1, wherein the crosslinking agent includes a polymerizable crosslinking agent having a (meth)acryloyl group.

8. The photosensitive resin composition according to claim 1, wherein the crosslinking agent includes a polymerizable crosslinking agent having an allyl group or a vinyl group.

9. The photosensitive resin composition according to claim 1, further comprising a thermal polymerization initiator.

10. A cured product of the photosensitive resin composition according to claim 1.

11. A semiconductor element comprising a redistribution layer containing a cured product of the photosensitive resin composition according to claim 1.
